(19) United States
(12) Patent Application Publication
(10) Pub. No.: US 2011/0236970 A1
(43) Pub. Date: Sep. 29, 2011

(54) CHAMBER OF A BIOREACTOR PLATFORM

(75) Inventors: Jacob Mollenbach Larsen, Copenhagen NV (DK); Ulrich Krühne, Copenhagen V (DK)

(73) Assignee: SMART BIOSYSTEMS APS, Copenhagen NV (DK)

(21) Appl. No.: 13/056,855

(22) PCT Filed: Jun. 18, 2009
(86) PCT No.: PCT/DK2009/050132

§ 371 Date: May 20, 2011

(30) Foreign Application Priority Data
Aug. 1, 2008 (DK) PA 2008 01061

(51) Int. Cl. C12N 5/07; C12M 1/00; C12N 5/071; C12N 1/20; C12N 1/16; C12N 1/14; C12N 5/04

(52) U.S. Cl. 435/348; 435/289.1; 435/395; 435/252.1; 435/255.1; 435/254.1; 435/420

(57) ABSTRACT

Disclosed herein is mesoscale bioreactor platform comprising an upwards open chamber for a biological cell, which chamber via a first port is in communication with a first channel for conducting an influent stream of a liquid into the chamber and via a second port is in communication with a second channel for conducting an effluent stream of a liquid away from the chamber, which chamber is provided with a closure comprising a water-immiscible liquid, and wherein said first channel is in fluid communication with a reservoir for a liquid and said second channel is in fluid communication with a waste container. Furthermore, a method for modifying the interaction of a content of a chamber with the surroundings is described as well as method of culturing a biological cell.

### FIELD OF THE INVENTION

This invention relates to a mesoscale bioreactor comprising a chamber for a biological cell, a channel for an influent stream and a channel for an effluent stream and a layer of a water-immiscible fluid as a closure on the chamber, wherein the channels are in fluid communication with a reservoir for a liquid and a waste container, respectively. The invention also relates to a method for modifying the interaction of the content of the chamber with the surroundings, and a method for culturing a biological cell. The mesoscale bioreactor is suited for culturing biological cells; it is especially suited for culturing mammalian cells, such as embryos or stem cells. More particularly it is suited for use in in vitro fertilisation procedures.

### PRIOR ART

The procedures currently employed in in vitro fertilisation (IVF) for embryo culture rely on culturing the embryos in Petri-dishes under static conditions. Such methodology is labour-intensive, as changes of growth media require a large degree of manual handling. Manual handling always introduces a risk of contamination, and moreover the static conditions do not provide much resemblance with in vivo conditions, as it is difficult to meet the changing needs of an embryo. In contrast to the current-day in vitro static conditions an embryo in vivo is exposed to a constantly changing environment, and the requirements of an embryo in one stage of development may be very different to those in another stage of development. The conditions existing in vivo at one stage of development may even be harmful to an embryo at a later stage of development.

The static conditions of a Petri-dish based system allow the use of open growth chambers, which may be directly accessed with a pipette or the like. For IVF-procedures open systems are convenient since they allow both replacement of buffers, and importantly, it is easy to remove the embryo after culturing. In order to minimise the risk of contamination and to prevent evaporation from the growth chambers, the aqueous culturing medium in the chamber is traditionally provided with a top layer of a water-immiscible liquid, e.g. paraffin oil, serving as a ‘lid’ or ‘closure’.

Some of the disadvantages of the static-based Petri-dish culturing system may be circumvented by culturing the embryo in a culturing system capable of perfusing the embryo with a growth medium appropriate for its developmental stage. Such a system should be sized appropriately to match the size of the embryo and to more closely resemble the conditions existing in vivo. Furthermore, it is important to work in small scale to minimise the consumption of expensive growth media typically required by such mammalian cells.

Many so-called microfluidic devices have now been described for conducting various types of analysis or for culturing cells. These devices are often created using various principles which are commonly inspired by the progress made in the 1970'ies with silicon-based technology for microelectronics. Examples of microfluidic applications are DNA-analyses involving principles such as the polymerase chain reaction for e.g. detection of single-nucleotide polymorphisms or assays for proteins using, e.g. capillary electrophoresis.

‘True’ microfluidic devices (e.g. with fluidic channels in the order of 100 μm diameter or less) do however suffer from a number of drawbacks, some of which are particularly pronounced for cell culturing devices designed for perfusion-type operation. As seen from the Hagen-Poiseuille equation (see below) the pressure drop in an e.g. 100 μm-channel with a flow becomes very large, putting high demands to a pump intended for operating at this scale, since such a pump must be able to precisely dispense very small volumes against a considerable back pressure. For this reason flows are often generated at this scale using so-called electroosmotic flow where a flow is created in a saline solution by exposing it to a large electrical potential. Such electroosmotic flow is however ill suited for systems involving live (mammalian) cells.

Another problem encountered in microfluidics is one related to the ‘connection to the outside world’. Most equipment employed in biological labs, such as pumps and analytical equipment, is so much larger than microfluidic equipment that integration between the two scales becomes problematic. Connection points for a tube as small as 250 μm-diameter (as is readily available) to a chip are difficult to handle for the lab worker, and moreover may quickly introduce dead volumes several times the size of the volume of the microfluidic system. This problem is especially important for perfusion-type cell culture devices where the operational complexity and the long residence times of fluids in tubes connected to a microfluidic system increases the risk of upstream contamination. In the case of culture of mammalian embryos the culture time can amount to five days or more.

For bioreactor systems working at small scales it is of course possible to switch between different growth media and conditions according to a predetermined sequence of events. However, in order to more fully optimise the growth conditions of the cells in a bioreactor the bioreactor may be equipped with appropriate sensors which communicate with a computer or similar capable of sending commands to actuators of the bioreactor. This way a feedback system may be created to respond to changes in the environment to e.g. maintain constant environmental conditions.

Numerous examples of biochemical or biological microfluidic devices have been described in the literature. In general, such devices are aimed at obtaining data from a sample taking advantage of the fast diffusion rates existing at microscale to quickly obtain data from even very small sample volumes. Analysis of nucleic acids, such as DNA or RNA, is particularly advantageous since the robust nature of nucleic acids allows liquids containing the nucleotides to be manipulated using electroosmotic flow. However, microscale fermenters have also been described in which microbial cells, e.g. bacteria, may be grown and observed or otherwise analysed while being exposed to various experimental conditions. See for example WO2005/123258 or WO2007/044699. A typical advantage provided by such systems will be that only very small volumes of sample liquids, possibly containing expensive test compounds, will be necessary to induce and study an effect on cells.

Most microfluidic devices described to date are, however, analytical devices aimed at providing abstract data about cells or biological compounds in sample liquids in the devices. The aim of IVF procedures will in contrast be the embryo obtained in the culturing process. Therefore, a device designed for perfusing a cell, such as an unfertilised or a fertilised oocyte, should provide easy access to the culturing chamber in order to allow the cell to be placed in the chamber, and especially also to be gently removed after the culturing period. This feature is not necessary in fluidic devices designed only for data acquisition where appropriate sensors may be integrated into the device allowing data to be extracted from the system without physically removing the cells.

As discussed below some steps have been taken to approach the above problem.

WO2007/047826 describes a microfluidic cell culture device, which employs an oil overlay layer to prevent evaporation of liquid from a microfluidic chamber and to allow access to a growth chamber in the device. The devices of WO2007/047826 may contain optical, electrical or electromechanical sensors to determine states or flow characteristics of elements of the microfluidic device. The device contains a funnel-shaped growth chamber and a reservoir connected via a first microchannel in the bottom of a PDMS substrate comprising the chambers. The reservoir and the growth chamber are further connected via a microchannel positioned above the first microchannel. With the aid of a membrane created from an elastomeric material and a so-called pin actuating device it is possible to create a peristaltic movement of liquid between the chambers. Thus, when fluid is moved peristaltically from the reservoir to the growth chamber via the bottom channel, the oil layer on the aqueous fluid in the growth chamber will be pushed via the upper channel into the reservoir and thereby retain a mass balance between the two chambers.

This peristaltic movement may be used to create a “back and forth-type of fluid supply wherein the fluid level in the well increases and then decreases cyclically”. However, the use of outside supplies of liquids is also suggested to apply liquid to the growth chamber of the device of WO2007/047826. Considering the “mass-balance-buffering” effect of the dual-channel design it is unclear how the design may be modified to use such external liquid supplies, and the devices seem ill suited for conducting long-term perfusion type growth experiments, as there is a need to use an outside supply of fluid. Thus, such a system is mainly of use when only two chambers are included in the fluidic system. In particular, this design is of little use when two or more reservoir chambers supply the same culturing chamber in a design where all the chambers are thus not serially connected.

WO2006/089354 describes a device for use in culturing a cell, in particular for IVF. The device comprises at least one upwards open culture chamber and a fluid reservoir, wherein the culture chamber is in fluid connection with the fluid reservoir. The medium of the cell culture chamber may be covered by a cell culture oil such as a paraffin-based oil to minimise evaporation. The cell culture chamber further has a tapered side wall. The fluid reservoir is connected to the culture chamber via an aperture in the culture chamber. The aperture is smaller than the diameter of the cell to be cultured such that the cell is maintained within the cell culture chamber.

The cell culture medium is injected into the fluid reservoir. From the fluid reservoir, the cell culture medium flows, preferably by capillary flow or by applied pressure difference via a fluid path to the aperture of the cell culture chambers and subsequently fills the cell culture chambers. The fluid level in the cell culture chambers will typically depend directly on the injected fluid volume. The fluid levels may be equilibrated by e.g. gravity. Thus, for example when a volume of liquid is injected into the reservoir the liquid will flow into the culturing chamber until the liquid levels in the two chambers are equal.

The fluid reservoir of WO2006/089354 can be used for both the ingress and egress of fluid from the culture chamber. In operation culturing medium can be added directly to the culture chamber of WO2006/089354 and excess liquid may be removed from the culture chamber by aspiration of liquid from the reservoir. It thus appears that the system described in WO2006/089354 is ill-suited for perfusive operation, in particular for long-term perfusive operation. The culture chamber of WO2006/089354 lacks a dedicated fluid inlet and a dedicated fluid outlet. A lack of such dedicated functions make it difficult to predict and control the conditions existing in the culture chamber, and also analysis of effluent fluid from the culture chamber is problematic since effluent fluid in the device of WO2006/089354 will inevitably be mixed with fresh medium. While a flow of liquid can be conducted through the chamber of WO2006/089354, the culture chamber does not have both an inlet and an outlet channel. Therefore the system appears ill-suited for perfusing the culture chamber, and in particular a steady state of the liquid level in the chamber could not be achieved.

Despite the efforts discussed above a system has yet to be described to solve the problems of designing a simple fluidic device intended for perfusion type operation on a scale and time appropriate for mammalian cells, such as embryos, where the growth chamber may readily be accessed during operation. It is an aim of the present invention to provide an upwards open chamber, which may be accessed physically during operation while retaining a separation between the liquid in the chamber and the ambient surroundings; this separation serves to prevent evaporation of solvents from the chamber and simultaneously prevent that the liquid in the chamber is contaminated with particles, in particular microbial germs or pathogens. An upwards open chamber may furthermore allow gases, such as O₂ or CO₂ to diffuse into liquid in the chamber, providing additional means to control the conditions in the chamber, such as the pH. Such a chamber is suited for culturing mammalian oocytes and embryos taking into account the different requirements to growth conditions during the development of the embryo as well as the period of time necessary for such culture, and further taking into account that it should be possible to access the chamber to place and remove cells from the chamber during perfusion of the chamber.

### DISCLOSURE OF THE INVENTION

The present invention relates to a mesoscale bioreactor platform comprising an upwards open chamber for a biological cell, which chamber via a first port is in communication with a first channel for conducting an influent stream of a liquid into the chamber and via a second port is in communication with a second channel for conducting an effluent stream of a liquid away from the chamber, which chamber is provided with a closure comprising a water-immiscible liquid, and wherein said first channel is in fluid communication with a reservoir for a liquid and said second channel is in fluid communication with a waste container. Thus, the present invention describes a bioreactor platform with a chamber for a biological cell. Bioreactor platforms will typically comprise a number of chambers serving as culturing chambers for biological cells, or for carrying out biological or biochemical reactions, such as culturing cells, hybridising nucleic acids or conducting enzymatic reactions; biological or biochemical entities taking part in such reactions may be immobilised on a surface of the chamber or may be freely suspended in a liquid in the chamber. Chambers in a bioreactor platform may also serve as reservoirs for liquids, e.g. buffer or medium containers, and bioreactor platforms will commonly comprise channels for conducting liquids between the chambers. Likewise bioreactor platforms commonly comprise waste containers. According to this invention any of these chambers, i.e. chambers for a biological cell, reservoirs for a liquid and waste containers may be upwards open, and they may be provided with a closure comprising a water-immiscible liquid.

The chamber contained in the mesoscale bioreactor platform of the invention is particularly suited where it is of interest to be able physically to access the chamber in a convenient manner. The upwards open chamber is provided with a closure of a water-immiscible liquid layered on top of an aqueous liquid in the chamber. The water-immiscible liquid will form a generally homogeneous phase in contact with a sidewall of the chamber defining the perimeter of the open surface of the chamber, thereby substantially preventing evaporation of liquid from the chamber and preventing that the aqueous liquid is contaminated with particles, e.g. microbial germs or pathogens, from the ambient surroundings of the bioreactor platform. The closure will also control the evaporation of solvents and other volatile compounds such as CO₂ and O₂. Some of the components in the aqueous liquid or media will effectively be hampered in escaping the chamber, such as water vapour, while other components may be exchanged over the closure, such as CO₂ and O₂. By controlling the transport of CO₂ over the closure of water-immiscible liquid the pH may be maintained at a relevant level. Thus, the water-immiscible liquid can be said to provide a semi-pervasive closure for the chamber.

Appropriate water-immiscible liquids are commonly transparent to visible light, which further allows cells in the chamber to be observed visually, e.g. by microscope. Being fluid, the water-immiscible phase may be readily penetrated with e.g. a pipette or the like, thus allowing access to the chamber and its contents, so that e.g. fertilised oocytes may be positioned in the chamber for culturing and gently removed after culturing.

The chamber contained in the mesoscale bioreactor platform of the invention is in communication with a channel via a port. By applying a positive relative pressure to the upper surface of the water-immiscible phase a liquid in the chamber may be pushed out of the chamber, thereby creating an effluent stream or flow. Likewise, a flow may be created by applying a negative relative pressure to the channel, so that a liquid in the chamber is aspirated out of the chamber. The water-immiscible phase may thus be said to constitute a flexible lid for the chamber. The mesoscale bioreactor platform of the invention comprises a chamber with a first port in communication with a first channel for an influent stream of a liquid into the chamber and a second port in communication with a second channel for an effluent stream of a liquid away from the chamber. These first and second channels will allow a liquid to be applied to and removed from the chamber, so that a steady state of the liquid level relative to e.g. the bottom of the chamber can be maintained. The first channel is in fluid communication with another chamber or reservoir containing a liquid, and this liquid may be aspirated or dispersed from the reservoir into the chamber. This operation may be employed to fill the chamber or retain the steady state. When the chamber is also fitted with a second channel for an effluent stream as well as a first channel for an influent stream, it may be employed for perfusive operation in e.g. IVF-procedures. The chamber may in the bottom surface comprise a depression for retaining the biological cell, such as for IVF and other procedures, e.g. culturing of other biological cells. In some embodiments a single chamber comprises multiple such depressions. When multiple depressions are present in a single chamber, the depressions may be connected serially, in parallel or in a combination of serial and parallel, with one or more channels for conducting a liquid between the depressions.

The chambers contained in the mesoscale bioreactor platform of the invention will commonly be formed in a substrate, which may be made from any convenient material, such as a polymer, a glass, a metal, a ceramic material or a combination of these. The substrate will define a bottom surface and a sidewall of the chamber; when viewed from above the sidewall may form a perimeter for the chamber, which is round, square, polygonal, or oblong, etc.; the perimeter is preferably round. The substrate will have an upper surface, and the upper surface surrounding the perimeter of the chamber may be oleophobic or superoleophobic to prevent the water-immiscible liquid from spreading on the upper surface of the substrate. An oleophobic or superoleophobic surface will provide a large contact angle, e.g. larger than 90°, for the interface between the water-immiscible liquid and the surrounding air on the substrate surface. A large contact angle is an indication that it is energetically disadvantageous for the water-immiscible liquid to spread on the substrate surface, and that the water-immiscible liquid will instead form a convex meniscus on an aqueous liquid in the chamber.

Biological or biochemical reactions normally take place in aqueous environments, and therefore in one embodiment the chamber for a biological cell comprises an aqueous liquid forming a lower phase in the chamber, so that the water-immiscible liquid forms an upper phase. The water-immiscible phase serves as a closure for the chamber to prevent evaporation and contamination of the aqueous liquid. In order to supply and remove aqueous liquid to or from the chamber, respectively, the lower aqueous phase preferably covers the first port and the second port of the chamber. This positioning of the ports and the aqueous liquid relative to each other will ensure that the water-immiscible phase can be retained on the aqueous liquid as a closure.

The bioreactor platform is preferably of mesoscale meaning that chambers and channels of the bioreactor platform will be sized appropriately for moderate numbers of biological cells, such as are used in in vitro fertilisation (IVF) procedures, for which the bioreactor platform of the invention is particularly suited. The bioreactor platform of the invention may comprise two or more chambers for a biological cell, e.g. a first chamber in fluid communication with a second chamber via the channel for conducting an effluent stream of a liquid away from the first of the chambers. The chamber forming a reservoir for a liquid, e.g. media, buffers or the like, may comprise a closure of a water-immiscible liquid and a channel leading to a culture chamber likewise provided with such a closure. One or more of these chambers, preferably the culture chamber, may be in fluid communication with a waste container that is also comprised in the bioreactor platform. The waste container will preferably also comprise a layer of a water-immiscible liquid, although in certain embodiments the waste container does not comprise a layer of a water-immiscible liquid.

In one embodiment the bioreactor platform also comprises a means to provide a liquid driving force to move a liquid via said first channel into the chamber for a biological cell and/or via said second channel away from the chamber for a biological cell, e.g. from the reservoir to the chamber for a biological cell and to the waste container via one or more of the channels. Thus, liquid may be driven from a chamber serving as a reservoir to a chamber serving as a culture chamber. As the bioreactor platform also comprises a waste container the liquid may further be driven to the waste container from the culture chamber. A liquid driving force may be provided by applying a positive relative pressure to the reservoir to disperse the liquid into the culture chamber, and optionally further into a waste container. Alternatively, a negative relative pressure applied to the channel for the effluent stream from the culture chamber will create the same effect: move liquid from the reservoir to the culture chamber. The negative relative pressure may also be applied to the waste container. Means to provide a liquid driving force may also be integrated into the bioreactor platform, e.g. in the form of a peristaltic function acting on a channel. In general, the means to provide a liquid driving force is selected from dispersing liquid into or aspirating liquid out of a chamber using an integrated or external pump, applying a positive relative pressure to the upper surface of a liquid in a chamber, applying a negative relative pressure to the upper surface of a liquid in a chamber, adjusting the level of the upper surface of the liquid in a first chamber to a higher level than the level of the upper surface of the liquid in a second chamber relative to a horizontal plane or any combination of these.

When the chambers of the bioreactor platform are upwards open, a liquid driving force may also be provided from differences in the horizontal position of the liquid surfaces in the chambers relative to each other. In one embodiment, wherein the chambers comprise an aqueous liquid the upper surface of the aqueous liquid in a first chamber, e.g. a reservoir, is at a higher level relative to a horizontal plane than the upper surface of the aqueous liquid in a second chamber, e.g. a chamber for a biological cell, relative to the horizontal plane. This provides a possibility to create a siphoning effect to move liquid from the first chamber into the second chamber via the channel for the effluent stream for the first chamber. The flow rate from one chamber to the next will be guided by the difference in height between the upper liquid surfaces, and also by any resistance to the flow resulting from the dimensions and materials of the channels. The different principles for providing a liquid driving force may also be combined. Thus, the siphoning effect may be combined with an integrated pump, or positive or negative relative pressures may be applied as discussed above to further or oppose the siphoning effect. The siphoning effect will be especially suited when the bioreactor platform comprises at least three chambers, e.g. one or two chambers according to the invention serving as (a) reservoir(s) for a liquid and a culture chamber, respectively, and a waste container, which may or may not be provided with an upper layer of a water-immiscible phase. In this embodiment the upper liquid surface of the reservoir will be higher than that of the culture chamber, the surface of which will in turn be higher than the upper liquid surface of the waste container. This may ensure that a steady state of the level of an aqueous liquid in the culture chamber is retained during operation of the bioreactor platform. The bottoms of the chambers may also follow the same pattern, i.e. with that of the reservoir being above that of the culture chamber, which in turn is above that of the waste container. Other parameters than the liquid levels in the chambers, which may influence the operation will be the flow resistance of the channels, as defined by the channel dimensions, and also the sizes of the surface areas.

The bioreactor platform may also contain multiple chambers with a reservoir function and/or multiple chambers for culturing biological cells. When multiple such culture chambers are present they may be arranged in one or more groups. The chambers in one group may be serially connected with channels for liquid streams, and the groups may be connected in parallel with channels for liquid streams. When the bioreactor platform is designed to employ the siphoning effect as discussed above, the bioreactor platform may also comprise multiple culture chambers.

In another aspect, the present invention relates to a method for modifying the interaction of a content of a chamber with the surroundings comprising the steps of:

providing a mesoscale bioreactor platform according to the invention;

applying an aqueous liquid to the chamber of the mesoscale bioreactor platform so that the aqueous liquid covers the first and the second ports;

applying a water-immiscible liquid of a density lower than that of the aqueous liquid in the chamber to form a lower aqueous phase and an upper phase comprising the water-immiscible liquid;

inducing a flow of the aqueous liquid into said chamber via said first channel and inducing a flow of the aqueous liquid away from said chamber via said second channel.

Any interaction between the content, e.g. a cell, a buffer or medium component, a liquid etc., of a chamber and the surroundings is appropriate for modification according to the method of the invention. In one perspective, the water-immiscible layer provides a hindrance to passage of undesired components, e.g. pathogenic germs, particulate contaminants or the like, into the chamber and therefore the interaction is modified by isolating the contents of the chamber from contamination from the ambient surroundings. The interaction may also be modified by changing or adjusting other conditions existing in the chamber and utilising the ability of the water-immiscible layer to form a hindrance to evaporation of liquid or diffusion of heat. Thus, when the temperature of the chamber is increased or decreased the water-immiscible layer will provide an insulating layer to a liquid in the chamber allowing control of the temperature. Likewise, the water-immiscible layer may prevent evaporation of liquid from the chamber. For some biological operations, such as IVF-procedures, it is necessary to have physical access to a growth or culture chamber with cells. In this case, the embryo formed from a fertilised oocyte is the product of interest of the procedure. Therefore, it must be possible to remove the embryo from the culture chamber. Moreover, the exact identity of a fertilised oocyte to be cultured is important, so that a convenient method of positioning the fertilised oocyte in the culture chamber is likewise of interest. For a bioreactor platform to be operated under perfusive conditions, i.e. with a liquid flow passing through the culture chamber, it has been suggested to employ a closable member, such as a lid to provide access to the chamber. However, when a lid is used it is necessary to interrupt the flow to gain access to the chamber, and moreover, when the lid is open the contents of the chamber are at risk of contamination with potentially pathogenic entities. A lid will also add to the complexity of the design making the bioreactor platform expensive.

The present inventors have surprisingly found that a layer of a water-immiscible liquid placed on top of an aqueous liquid in a chamber of a bioreactor platform may be employed instead of a lid to provide a closure for the chamber, even when a flow of liquid is being perfused through the chamber. Thus, the invention further relates to a method comprising the steps of:

providing a mesoscale bioreactor platform according to the invention;

applying an aqueous liquid to the chamber of the mesoscale bioreactor platform so that the aqueous liquid covers the first and the second ports;

applying a water-immiscible liquid of a density lower than that of water on the aqueous liquid in the chamber to form a lower aqueous phase and an upper phase comprising the water-immiscible liquid;

inducing a flow of an aqueous liquid into said chamber via said first channel and inducing a flow of the aqueous liquid away from said chamber via said second channel, wherein the level of the aqueous liquid in the chamber is maintained at a steady state.

In yet another embodiment the method of the invention further comprises the step of controlling the pressure of a gas above the chamber relative to the pressure of the gas originating from the aqueous liquid in the chamber in order to control diffusion of the gas into or out of the aqueous liquid. The gas is preferably CO₂ or O₂.

The water-immiscible liquid may also be applied to the chamber comprising either a channel for an effluent flow, or both a channel for an effluent flow and a channel for an influent flow, before application of the aqueous liquid. This will not impair the function of the closure by the water-immiscible liquid, since the higher density of the aqueous liquid will ensure that the liquids are layered as intended.

In another aspect the invention relates to using a water-immiscible liquid as a closure for a chamber in a mesoscale bioreactor platform. Herein a mesoscale bioreactor platform comprising an upwards open chamber for a liquid and a first channel communicating with the chamber via a first port is provided prior to applying an aqueous liquid to the chamber of the mesoscale bioreactor platform so that the aqueous liquid covers the first port, applying a water-immiscible liquid of a density lower than that of water on the aqueous liquid in the chamber to form a lower aqueous phase and an upper phase comprising the water-immiscible liquid, inducing a flow of the aqueous liquid from the chamber into said first channel to create an effluent stream, and controlling the pressure of a gas above the chamber relative to the pressure of the gas originating from the aqueous liquid in the chamber in order to control diffusion of the gas into or out of the aqueous liquid.

The closure formed by the water-immiscible liquid allows gases to diffuse through it. However, the water-immiscible liquid does represent a hindrance to this diffusion and to evaporation of solvent through the layer. Thus, when there is a gradient in the pressure of the gas above the chamber relative to the pressure of the gas originating from the aqueous liquid, the gas will diffuse according to this gradient. But at the same time the layer of the water-immiscible liquid prevents evaporation of solvent from the chamber, so that the osmolarity of the can be maintained.

In the present invention it is preferred to control the pressure of CO₂ above the chamber. A high relative pressure of CO₂ will force the CO₂ into the aqueous liquid, which in turn may lead to a decrease in the pH of the liquid. In contrast, a low pressure of CO₂ may allow CO₂ to diffuse out of the liquid thereby increasing its pH. Thus, by controlling the pressure of CO₂ above the chamber it is possible to modify the pH of the aqueous liquid in the chamber. The concentration of the gas above the chamber is preferably air premixed with e.g. 2-10% CO₂, preferably 5%. It may also be a trigas with 2-20% O₂. The total pressure above the chamber may be increased slightly compared to normal, atmospheric pressure, and the pressure of CO₂ may be calculated from its concentration and this total pressure.

In a further aspect the invention relates to a method of culturing a biological cell comprising the steps of providing a mesoscale bioreactor platform according to the invention, placing the biological cell in the chamber for a biological cell and perfusing the cell with an aqueous liquid. The biological cell may be a mammalian, bacterial, yeast, fungal, plant, or insect cell. When the cell is mammalian the cell may be e.g. a spermatozoon, oocyte, embryo, stem cell, monocyte, dendritic cell, or a T-cell, although the method is not limited to these cells. In a certain embodiment the cell is cultured for three days or more.

### BRIEF DESCRIPTION OF THE FIGURES

In the following the invention will be explained in greater detail with the aid of examples of embodiments and with reference to the schematic drawings, in which

FIG. 1 shows a side view of an upwards open chamber.

FIG. 2a shows a side view of an upwards open chamber of a mesoscale bioreactor platform according to the invention, wherein the sidewall of the chamber is oleophilic.

FIG. 2b shows a side view of an upwards open chamber of a mesoscale bioreactor platform according to the invention, wherein the sidewall of the chamber is oleophobic.

FIG. 3 shows a side view of a mesoscale bioreactor platform according to the invention.

FIG. 4 shows a side view of a mesoscale bioreactor platform according to another embodiment of the invention.

FIG. 5a shows a perspective view of mesoscale bioreactor platform of the invention.

FIG. 5b shows a perspective wireframe view of mesoscale bioreactor platform of the invention.

FIG. 6 shows a curve for the pH of the aqueous liquid in an upwards open chamber in a mesoscale bioreactor platform of the invention.

### DETAILED DESCRIPTION OF THE INVENTION

The present invention relates to a mesoscale bioreactor platform comprising an upwards open chamber for a biological cell, which platform comprises a channel for an influent stream, a channel for an effluent stream and a layer of a water-immiscible fluid as a closure on the chamber as well as a reservoir for a liquid and a waste container. The invention further relates to a method for modifying the interaction of a content of a chamber with the surroundings employing a layer of a water-immiscible fluid as a closure of a chamber in a mesoscale bioreactor. A method of culturing a biological cell is also comprised in the invention.

The term “bioreactor platform” or “bioreactor” of the present invention cover systems and devices suited for culturing biological cells. The disclosed chamber and the bioreactors are especially suited for mammalian cells. In a preferred embodiment the mammalian cells are cells related to in vitro fertilisation (IVF), and the cells will comprise spermatozoa, oocytes, and/or embryos. However, as will be obvious to those skilled in the art the bioreactor may also be useful for other mammalian cell types, such as stem cells or cells of the immune system, such as monocytes, dendritic cells, T-cells and the like. In a preferred embodiment the mammalian cells are human cells. Furthermore, an upwards open chamber or a mesoscale bioreactor as disclosed in the present invention may also be of utility in the culturing of cell types other than mammalian cells. For example, bacterial, yeast, fungal, plant, or insect cells may also be cultured in the bioreactor disclosed herein.

Bioreactors will comprise various types of chambers, such as reservoirs for liquids, e.g. buffers or media, culture chambers (i.e. chambers for biological cells) and/or waste containers. In the context of the present invention a “chamber” will generally be upwards open. This means that the chamber is defined by a bottom surface and a sidewall; the sidewalls may be substantially vertical, or the chamber may be downwards tapered. The chamber may also comprise a “ceiling” placed vertically above the bottom, though such a ceiling will not fully cover the surface of a liquid in the chamber. A waste container is a chamber not comprising a channel for an effluent stream, so that the liquids being perfused through chambers in a bioreactor platform will eventually be collected in the waste container. Thus, the waste container collects spent medium from the culture chamber. However, liquid may also be removed from the waste container, e.g. in order to analyse the liquid, or to adjust the volume of liquid in the waste container.

By being upwards open, the culture chamber provides convenient physical access to the chamber. In this context the term “physical access” means that a tool may be inserted into the liquid in the culture chamber to manipulate the contents of the culture chamber. This manipulation may be to insert or remove one or more cells from the culture chamber, or it may involve manipulations of cells already present in the culture chamber. Such manipulation is conveniently obtained using a tool, such as a disposable pipette.

Biological and biochemical reactions will most often take place in aqueous solutions. In the context of the present invention an “aqueous liquid” is a liquid containing solvents, which may be mixed with water. Most commonly the aqueous liquid will only comprise water as a solvent, but for certain operations solvents such as methanol, ethanol, propanol, DMSO, glycerol etc. may also be present. The aqueous liquid will also normally contain salts and buffer components, such as NaCl, phosphates, as well as nutrients or other components, such as dissolved oxygen (O₂), carbon dioxide (CO₂), glucose, vitamins, metabolites, specific proteins or enzymes, etc. Appropriate media for use in IVF-procedures are well known in the art, as represented by those available from MediCult A/S (Jyllinge, Denmark).

In contrast to the aqueous liquid a “water-immiscible liquid” comprises components that cannot be mixed with or dissolved in water. These components may be oils or fats of a biological source, such as plant oils or the like, or mineral oils or synthetic oils, such as paraffin oil. The water-immiscible liquid preferably comprises paraffin oil. Water-immiscible liquids typically have a lower density than that of water, so that when such a liquid is placed on an aqueous liquid a two-phased system will be formed with a layer of the water-immiscible liquid on top of the aqueous liquid. The amount of water-immiscible liquid used should be sufficient to fully cover the surface of the aqueous liquid interfacing the air of the ambient surroundings. When fully covering the surface of the aqueous liquid, the water-immiscible liquid will be in contact with the perimeter of the chamber as defined by the substrate of the bioreactor platform. In this instance, the water-immiscible liquid can be said to provide a closure to the chamber. An appropriate thickness of the layer of water-immiscible liquid is between 0.5 to 3 mm, such as between 1 mm and 2 mm, e.g. about 1 mm or about 2 mm.

By “closure” is meant that the water-immiscible liquid will prevent evaporation of the aqueous liquid or other components therein, such as CO₂, from the chamber and further prevent particles, such as microbial germs or pathogens, from entering the aqueous liquid. It is an important characteristic of a closure formed by a water-immiscible liquid on an aqueous liquid in a chamber that gases may diffuse through the water-immiscible liquid. Thus, for example the direction of diffusion of a gas, e.g. CO₂, will depend on the pressure of the gas in the air above the chamber and the concentration of the gas in the aqueous liquid. Thereby the pH of an aqueous liquid may be controlled by adjusting the pressure of CO₂ above the chamber. For example, by increasing the CO₂-pressure CO₂ will be forced into the aqueous liquid and lower the pH; likewise a low CO₂-pressure will lead to evaporation of CO₂, thereby increasing the pH. In addition the closure may function as a heat-insulating layer, which may facilitate maintaining a constant temperature, such as 37° C., of the aqueous liquid in the chamber. However, the closure provided by the water-immiscible liquid does not prevent physical access by an operator to the contents of the chamber. Thus, an operator may penetrate the water-immiscible liquid, with e.g. a pipette, and gain access to the chamber. Upon removal of the pipette the water-immiscible liquid will again form the closure.

In the context of this invention the term “mesoscale” is intended to cover a range of sizes where the smallest dimension of channels is in the range from around 100 μm to around 3 mm, although the channels may also contain constrictions. Likewise the culture chamber may be of a depth of around 500 μm to around 5 mm or more, and the largest horizontal dimension may be from around 1 mm to around 50 mm. In one embodiment the upper surfaces of the aqueous liquids in the chambers relative to a horizontal plane are at different levels. This will be reflected in the depth of the chambers relative to the upper surface of the substrate comprising the chambers. The size of reservoirs must be sufficient to supply cells cultured under perfusion conditions with appropriate media through-out the culturing. Bioreactor systems in the mesoscale size range are particularly convenient where it is of interest to be able to physically manipulate the cells in a culture chamber, and to quickly be able to locate an individual location containing cells, such as an embryo, based on their origin. Furthermore, it can generally be said that fluids in mesoscale fluidic systems will be flowing under laminar conditions, and fluidic systems with channels or chambers different from those defined above may well be described as “mesoscale” as long as fluids contained in the systems flow under laminar conditions.

At mesoscale and smaller scale the pressure drop experienced by a fluid moving through a channel may become highly significant as can be estimated from the Hagen-Poiseuille equation for a Newtonian fluid:

$$Δ P = \frac{Δ x 8 μ Q}{π {r}^{4}}$$

Here ΔP is the pressure drop over a length of channel, Δx, of radius r of a fluid of dynamic viscosity μ, flowing at volumetric flow-rate Q. Thus, from the equation may be defined a flow-resistance parameter, Δx/r⁴, for a given channel.

In a certain embodiment the largest horizontal dimension of the culture chamber is in the range from around 2 to around 6 mm. In another embodiment the largest horizontal dimension of the culture chambers is in the range from around 20 to around 30 mm. Within the range of flow-rates typically employed in the mesoscale bioreactors of the invention the liquids will be moving in an essentially laminar flow.

The bioreactor platform of the present invention is suited for operation under perfusion conditions, and the conditions outlined below may be employed in the methods of the invention. In this context the terms “perfusion” or “perfuse” mean that a generally continuous flow is applied to a culture chamber of the device. This continuous flow is not limited to a certain flow-rate, but during the course of an experiment with a bioreactor platform of the present invention several different flow-rates may be employed. Suitable flow-rates are from around 1 μL/h to around 200 μL/min or more, although even lower flow-rates may also be used. The flow may be generated in pulses; at a low number of pulses at a small volume per pulse, such as 1, 2, 3 or up to 10 pulses of e.g. 0.5 μL, 1 μL etc., per time interval, such as per minute or per hour, e.g. 1 pulse of 1 μL per hour, the flow will in practice perform as a continuous flow. It should be emphasised that the flow may also be stopped if necessary, e.g. for performing various operations involving the contents of the culture chamber(s). Furthermore, intermediate operation allowing rest to the biological cells is also contemplated.

Upwards open chambers for use in bioreactor platforms designed for perfusion operation or otherwise involving a flow out of and/or into the chamber pose a challenge compared to conventional fluidic systems with upwards closed chambers, optionally connected to an external reservoir for liquid. The behaviour of liquids in closed chambers is easily predicted, since the liquid can only leave the chamber via any channels or conduits communicating with the chamber. In contrast, an upwards open chamber communicating with a channel allows liquid in the chamber to leave via either the channel or the upper surface of the chamber as defined by the substrate housing the chamber. The open nature makes it necessary to more carefully consider pressure differences between chambers in fluid communication to ensure control of the fluid flow between the chambers.

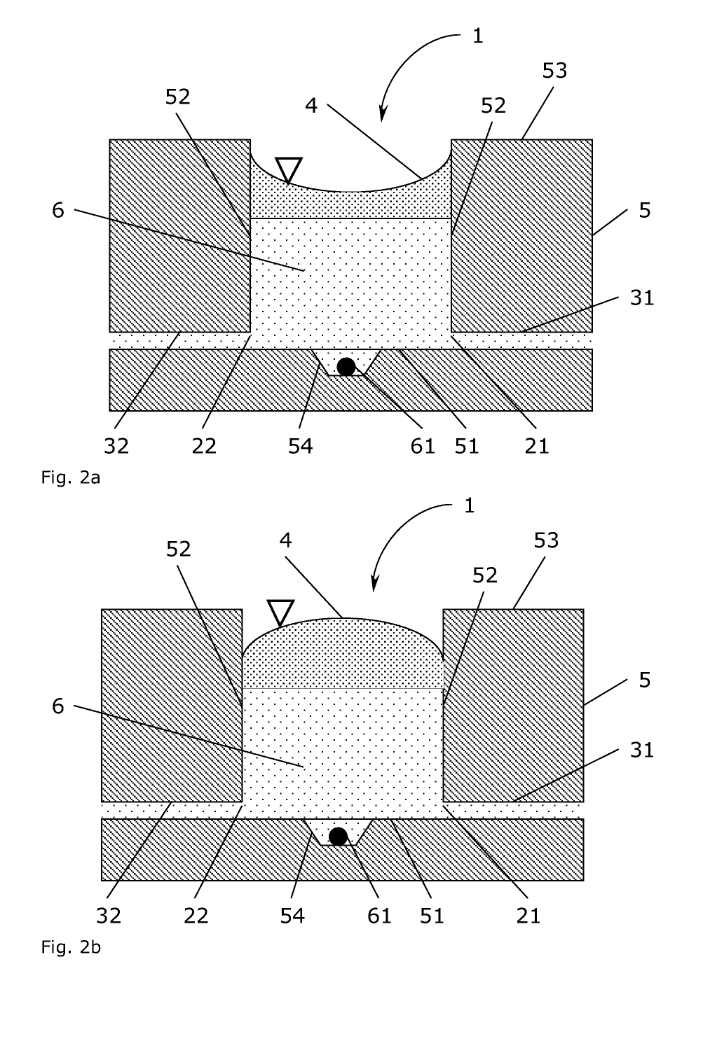

The present inventors have now found that a layer of a water-immiscible liquid can be employed to form a closure on an upwards open chamber for a bioreactor platform for isolating the contents of the chamber from contamination from the ambient surroundings to prevent evaporation and contamination of aqueous liquids in the chamber. The chamber of a mesoscale bioreactor platform of an embodiment of the invention is illustrated in FIG. 2. Thus, the invention comprises a mesoscale bioreactor platform with an upwards open chamber 1 for a biological cell, which chamber via a first port 22 is in communication with a first channel 32 for conducting an influent stream of a liquid into the chamber 1, and wherein the chamber 1 is provided with a closure comprising a water-immiscible liquid 4. The chamber 1 also comprises a second port 21 in communication with a second channel 31 for an effluent stream of a liquid away from the chamber 1. The chamber 1 may be formed in a substrate 5 defining a bottom surface 51 and a sidewall 52 of the chamber 1, which substrate has an upper surface 53. The chamber 1 may further comprise an aqueous liquid 6 forming a lower phase, wherein the water-immiscible liquid 4 forms an upper phase, and wherein the lower aqueous phase covers the first port 22 and the second port 21. The bottom of the chamber may also comprise a depression 54 for retaining a biological cell 61 during culture.

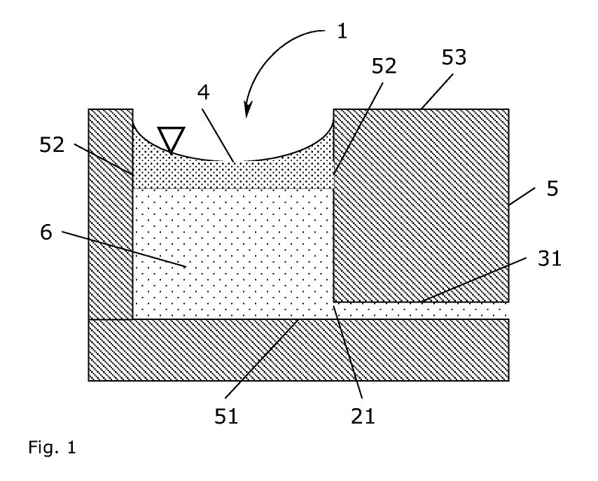

In another aspect the invention relates to a method of for modifying the interaction of a content of a chamber with the surroundings, e.g. isolating the contents of a chamber in a mesoscale bioreactor platform from contamination from the ambient surroundings. When a chamber has only a single outlet, such as is found for a reservoir for a liquid as illustrated in FIG. 1, an aqueous liquid 6 is applied to an upwards open chamber 1 of a mesoscale bioreactor platform housing the chamber 1 so that the aqueous liquid 6 covers the port 21; a water-immiscible liquid 4 of a density lower than that of water is then applied on the aqueous liquid 6 in the chamber 1 to form a lower aqueous phase and an upper phase comprising the water-immiscible liquid 4; a flow of the aqueous liquid 6 from the chamber 1 into the channel 31 is then induced to create an effluent stream. In an embodiment of the invention as shown in FIG. 2, a method of using a water-immiscible liquid as a closure for a chamber 1 comprising a first channel 32 communicating with the chamber 1 via a first port 22 and a second channel 31 communicating with the chamber via a second port 21 is described. Herein the aqueous liquid 6 is applied so that it covers the first port 22 and the second port 21, before application of the water-immiscible liquid 4 on the aqueous liquid 6 in the chamber 1 to form a lower aqueous phase and an upper water-immiscible phase. A flow of an aqueous liquid 6 into said chamber 1 via said first channel 32 and a flow of the aqueous liquid 6 away from said chamber 1 via said second channel 31 is then induced.

The partial pressure of gas, such as CO₂, above the upwards open chamber 1 with the water-immiscible liquid 4 is preferably controlled. The partial pressure of O₂ may also be controlled. The pressure of the gas above the water-immiscible liquid 4 is controlled relative to the pressure of the gas originating from the aqueous liquid in the chamber in order to control diffusion of the gas into or out of the aqueous liquid. Thus, when the pressure of the gas above the chamber is higher than the pressure of the gas from the liquid in the chamber, the pressure gradient will drive the gas to diffuse into the liquid. By controlling the pressure of CO₂ it is thereby possible to control the pH of the aqueous liquid in the chamber, since an increase in the CO₂-concentration will lead to a decrease in pH. The gas above the upwards open chamber 1 may be air or it may be air premixed with e.g. 2-10% CO₂, preferably 5%, and/or it may be a trigas with 2-20% O₂.

The water-immiscible liquid 4 may also be applied to the chamber prior to application of the aqueous liquid 6. The higher density of the aqueous liquid 6 will ensure that a two-phase system is formed with the water-immiscible liquid 4 forming an overlay layer on top of the aqueous liquid 6, which in turn will cover the ports 21 and 22 as appropriate. The water-immiscible liquid 4 will now form the desired closure on the chamber 1.

In its most simple form with only a single channel 31 for an effluent or influent stream, a flow of an aqueous liquid may be induced from the chamber 1 into the channel 31 by applying a positive relative pressure (as indicated with the triangle in FIG. 1) to the surface of the water-immiscible liquid 4, thereby pushing the aqueous liquid out of the chamber 1 via the channel 31. Likewise, a negative relative pressure may create an influent flow via channel 31. The pressure applied to the water-immiscible liquid 4 should be considered in relation to the pressure drop in the channel 31 as estimated e.g. from the Hagen-Poiseuille equation above. A flow may also be induced by applying a negative relative pressure to the channel 31. Such a negative relative pressure may be provided with the aid of a pump (not shown), which may be integrated with a bioreactor platform comprising the chamber 1 and the channel 31, or the pump may be located externally.

In contrast to this simple form, a fluidic system, such as the mesoscale bioreactor platform of the invention, with an open chamber 1 and a channel 32 for an influent flow of fluid to the chamber 1 and a channel 31 for an effluent flow of fluid from the chamber 1 has three different routes for the liquid to move along: the channels 31 and 32 and through the surface defining the open section of the chamber 1. This means that it may be a challenge to retain a steady state with a substantially constant liquid level in the chamber 1, i.e. to ensure that the amount of liquid leaving the chamber via the channel 31 is equal to the amount entering the chamber 1 via the channel 32. In this embodiment, a flow may be induced by applying a positive relative pressure to the channel 32 and a negative relative pressure to the channel 31 while at all times keeping into consideration the external pressure applied to the surface of the water-immiscible liquid 4 in the chamber 1. A steady state may e.g. be provided by having a liquid displacement function, such as a pump, at each of the channels 31, 32 so that the liquid flows may be controlled.

It is also possible to induce a flow of the aqueous liquid by applying either a positive relative pressure to the channel 32 for an influent stream or a negative relative pressure to the channel 31 for an effluent stream. When only a single liquid displacement function is applied the interfacial interactions between the water-immiscible liquid 4, the substrate 5 and the ambient air are parameters for consideration. In particular, the interaction between the water-immiscible liquid 4 and the sidewall 52 of the substrate 5 is of importance. In general it can be said that the surface characteristics of the sidewall 52 will determine the shape of the meniscus of the water-immiscible liquid 4. When the sidewall 52 of the chamber 1 is oleophilic (or hydrophobic) the meniscus will be concave as illustrated in FIG. 2a; in contrast an oleophobic (or hydrophilic) sidewall 52 will lead to formation of the convex meniscus shown in FIG. 2b. At the relevant scale of operation (i.e. chamber diameters between 1 and 50 mm, such as about 2.5 mm, about 8 mm, about 12 mm, about 14 mm, about 25 mm, etc.) the present inventors have found that the combined forces of adhesion between the water-immiscible liquid 4 and the sidewall 52 of the substrate 5, and the surface tension of the water-immiscible liquid 4 with the ambient air may provide a sufficient resistance to an effluent flow via the open surface of the upwards open chamber 1. Thus, when a flow of aqueous liquid 6 is created by dispersing aqueous liquid 6 into the chamber 1 by a positive relative pressure from channel 32, the closure provided by the water-immiscible liquid 4 will be held in place by the adhesion of the water-immiscible liquid 4 to the sidewall 52 and the surface tension between the water-immiscible liquid 4 with the ambient air, so that the liquid is dispersed out of the chamber 1 substantially only via channel 31. This effect is especially pronounced when the sidewall 52 is oleophilic.

In one embodiment the thickness of the layer of the water-immiscible liquid 4 (as indicated by the distance from the upper surface of the aqueous liquid to the bottom of the surface of a concave meniscus or the top of the surface of a convex meniscus) is between 0.5 to 3 mm, such as about 1 mm or about 2 mm. The thickness of this layer is normally not dependent on the diameter of the chamber comprising the water-immiscible liquid 4.

In another embodiment of the chamber 1 according to the invention, the upper surface 53 of the substrate 5 surrounding the perimeter of the chamber 1 is oleophobic or superoleophobic. This will augment the effectiveness of the closure provided by the water-immiscible liquid 4 by preventing the water-immiscible liquid 4 from spreading on the upper surface 53 of the substrate. Thus, an oleophobic or superoleophobic perimeter will make it energetically favourable for the water-immiscible liquid 4 not to spread, but instead attain a convex shape, and thereby maximise the enclosing capability of the water-immiscible liquid 4. The features of the different embodiments may be combined freely so that an oleophobic or superoleophobic perimeter may be used with liquid displacement functions acting on both channels 31 and 32.

In another embodiment of the invention the mesoscale bioreactor platform 8 comprises two or more chambers as described above. In this embodiment a first chamber or reservoir 11 is in fluid communication with a second chamber or culture chamber 12 via the channel 31 for conducting an effluent stream of a liquid away from the first of the chambers. The bioreactor platform further comprises a waste container 7, wherein at least one of the channels, e.g. channel 31′ of chamber 12, is in fluid communication with the waste container 7. In one embodiment the mesoscale bioreactor platform 8 further comprises means (not shown) to provide a liquid driving force to move a liquid from one of the chambers to another of the chambers and/or to the waste container 7 via one or more of the channels 31, 31′. The means to provide a liquid driving force may be integrated or may be external to the bioreactor platform 8.

The substrate 5 of the bioreactor platform may be capable of forming a substantially air-tight connection with a control unit (not shown), so that the air pressure above the surface of the water-immiscible liquid 4 may be controlled. Moreover, the control unit may also be constructed so as to make air-tight connections with the substrate 5 so that the air-pressure above the water-immiscible liquid 4 of separate chambers 11,12, optionally also 7, may be controlled independently. In one embodiment, the air pressure above chambers functioning as reservoirs for media or buffers may be controlled independently for each reservoir chamber. In another embodiment, the pressure above the waste container 7 may also be controlled independently. The air pressure above a chamber may be controlled with the aid of a pump, or the control unit may comprise a cylindrical chamber with a piston providing the function of a syringe for controlling the pressure.

The pressure of a chamber may also be increased or decreased by respectively adding or removing a water-immiscible liquid, thereby modifying the mass of the water-immiscible liquid 4.

Appropriate external pumps for providing a liquid driving force may be a peristaltic pump, a piston pump, a syringe pump, a membrane pump, a diaphragm pump, a gear pump, a microannular gear pump, or any other appropriate type of pump. Integrated pumps may be peristaltic pumps, piston pumps, pumps driven by electrolytically produced gas, or other types. The mesoscale bioreactor platform may also comprise valves, e.g. one-way-valves, to aid in directing a flow through the mesoscale bioreactor platform.

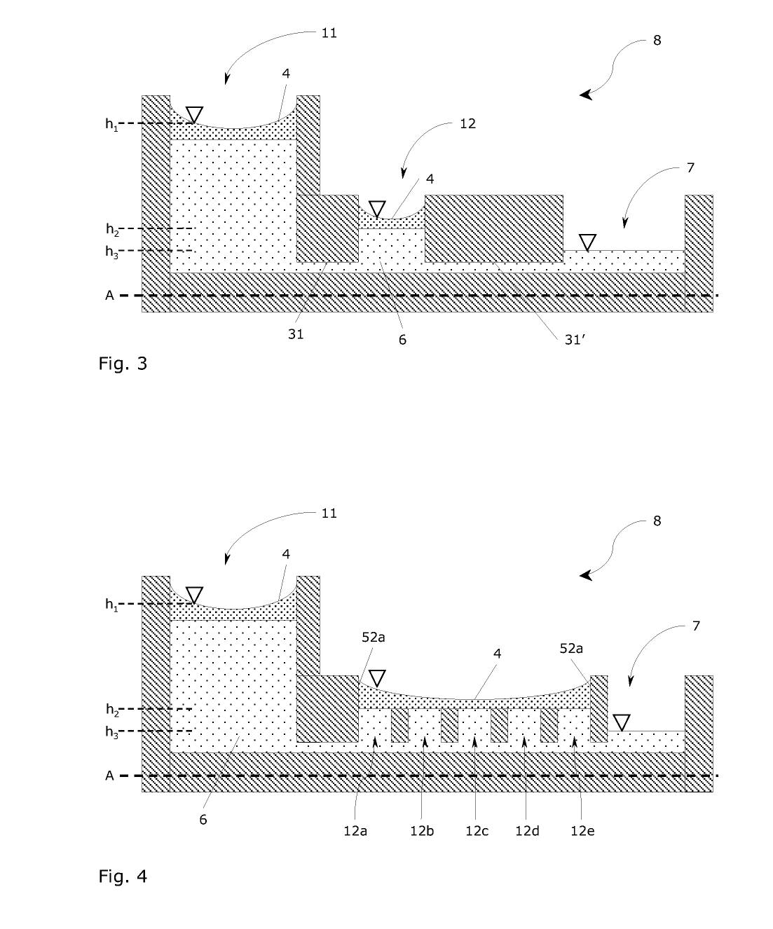

The liquid driving force may also be provided using gravitationally driven flow. In this embodiment, as depicted in FIG. 3, the chambers 11, 12 and the waste container 7, comprise an aqueous liquid 6, wherein the upper surface of the aqueous liquid 6 in a first chamber 11 is at a higher level h₁ relative to a horizontal plane A than the level h₂ of the upper surface of the aqueous liquid 6 in a second chamber 12 relative to the horizontal plane A. The levels of the upper surfaces of the layers of the water-immiscible liquids 4 in the chambers 11, 12 should follow the same pattern. As a waste container 7 is also present the surface of the aqueous liquid 6 will be at an even lower level h₃. The density of the aqueous liquid 6 as well as the mass/density of the water-immiscible liquids 4 combined with the different heights of the upper liquid surfaces and the force of gravity will create a driving force to move the aqueous liquid 4 in chamber 11 via channel 31 to chamber 12 and optionally from there into the waste container 7 via channel 31′. As long as the pressure difference between the chambers 11, 12 and optionally the waste container 7 is higher than the pressure drop caused by the resistance to the flow in channels 31, 31′ the aqueous liquid 6 will flow from chamber 11 to 12 and optionally waste container 7. This gravitationally driven flow may be controlled further by aspirating liquid from the waste container 7.

The flow of the aqueous liquid 6 may further be controlled by application of an external, positive or negative relative, pressure to chamber 11 and/or the waste container 7. For example, the substrate of the bioreactor platform at the location of chamber 11 may allow a substantially air-tight connection with a control unit around the perimeter of chamber 11, so that the pressure above the water-immiscible liquid in chamber 11 can be controlled, e.g. increased to disperse the aqueous liquid 6 into channel 31 and chamber 12. At all times the retainment capability of the water-immiscible liquid 4, discussed above, in chamber 12 will apply to prevent an effluent flow of the aqueous liquid 6 via the upwards open surface. This embodiment of the mesoscale bioreactor platform may also comprise integrated or external pumps, and the channels may be provided with valves. For example, a one-way valve in channel 31 will prevent a back-flow of aqueous liquid 6 from chamber 12 to chamber 11.

In a preferred embodiment, the mesoscale bioreactor platform comprises two reservoir chambers, one or more culture chambers and a waste container. A pump, such as a piston pump, is in fluid communication with the liquid in the waste container, so that a liquid flow in the mesoscale bioreactor platform may be generated by aspiration of liquid from the waste container via the pump. The flow from the reservoir chambers into the culture chamber(s) is controlled by making air-tight connections above the reservoir chambers, i.e. blocking a flow of gas or air into the reservoir chambers. Thus, The flow may be controlled to be from one of the reservoirs into the culture chamber(s) by blocking the flow of air to the other reservoir chamber. The mesoscale bioreactor platform may also comprise more than two reservoir chambers, for which the flow of air into the reservoir chambers may be blocked individually and independently.

The bioreactor platform may also comprise multiple chambers; for example a bioreactor platform may comprise a number, e.g. 2, 3 or more reservoir chambers for different culture media, which multiple chambers may be in fluid communication with a single culture chamber, so that a biological cell in the culture chamber may be supplied with different medium compositions from a single reservoir chamber or from a combined medium composition deriving from more than one reservoir chamber.

The mesoscale bioreactor platform of the present invention is not limited to a single chamber for culturing cells. Actually, in some embodiments the bioreactor platform comprises several such culture chambers, for example 10-20 culture chambers. These culture chambers may be arranged in one or more groups of serially connected culture chambers. Each group may be connected in parallel with chambers functioning as reservoirs. Thus, the platform may contain a single culture chamber, multiple culture chambers connected in a single series, multiple culture chambers connected in parallel, or groups of serially connected culture chambers where each group is connected in parallel.

Neither of the embodiments of the mesoscale bioreactor platform will comprise any direct contact between the water-immiscible liquids 4 in each of the chambers 11, 12. For each chamber 11 or 12 the water-immiscible liquid 4 will be separate from that on the other chamber, so that the water-immiscible liquid 4 can be said to independently serve as a closure on the respective chamber. When the mesoscale bioreactor platform employs multiple culture chambers the bioreactor platform may, however, be designed so that the culture chambers share a single closure formed by the water-immiscible liquid. In the embodiment shown in FIG. 4, a layer of water-immiscible liquid 4 is formed on the five culture chambers 12a-e. The water-immiscible liquid 4 is confined by sidewalls 52a, so that the cross-sectional area and the height of the water-immiscible liquid 4 are well defined. This is especially advantageous when the bioreactor platform 8 employs the siphoning effect as a liquid driving force as discussed above, although the liquid driving force may also be provided using other principles. Likewise, the bioreactor platform 8 is not limited to serially connected culture chambers as shown for chambers 12a-e in FIG. 4; the culture chambers may also be connected in parallel, or the bioreactor platform may comprise groups of serially connected culture chambers with the groups connected in parallel. When several culture chambers share a single closure formed by water-immiscible liquid 4 it is important that the water-immiscible liquid 4 enclosing the culture chambers is not in contact with the water-immiscible liquid 4 enclosing the reservoir chamber(s) 11, but that liquid transfer between the chambers 11 and 12, respectively, is transmitted via the aqueous liquid 6 via the channels.

The mesoscale bioreactor platform of the present invention is particularly suited for use in IVF-procedures. Likewise, the method of isolating the contents of a chamber in a mesoscale bioreactor using a water-immiscible liquid as a closure for a chamber in a mesoscale bioreactor platform is suited for IVF-procedures. Thus, in one aspect the invention relates to a method for culturing a biological cell, which is preferably a biological cell relevant for IVF-procedures, such as a spermatozoon, oocyte, embryo etc. These cells are preferably of human origin although cells of these types from other mammals are also within the scope of the invention.

A typical IVF-procedure involves the use of a bioreactor platform according to the invention. In this case, the bioreactor platform may contain a number of chambers functioning as reservoirs for different culture media. A reservoir will typically be a chamber with an effluent channel, but not containing a channel for an influent flow, connected directly to a culture chamber or the reservoir chamber may be connected to the culture chamber via a manifold to which effluent channels from several reservoir chambers are connected. A bioreactor platform according to the invention for use in IVF-procedures will normally have two or more reservoir chambers. Each reservoir chamber will be fitted with an overlay layer on the aqueous medium of a water-immiscible liquid functioning as a closure for the reservoir chamber. The bioreactor platform may, however, also comprise only one reservoir chamber.

In contrast, a culture chamber will normally have both an influent channel and an effluent channel, so that fresh medium may be provided from the reservoir(s) to the culture chamber and so that spent medium may be removed from the culture chamber via the effluent channel, which in turn will be in fluid communication with a waste container. As for the reservoir the culture chamber will have a water-immiscible liquid forming a protective closure on the aqueous liquid in the chamber.

The waste container will normally be integrated with the bioreactor platform, and like the reservoir chamber and the culture chamber it will normally comprise a water-immiscible liquid, although in some embodiments the waste container does not comprise a water-immiscible liquid. The waste container may also be located externally relative to the bioreactor platform with effluent streams being led to the container from the culture chamber via channels on the bioreactor platform and possibly also external tubing.

For an IVF-procedure, the water-immiscible liquids may be applied after filling the reservoirs and the culture chamber. However, it is also possible to apply aqueous media to the platform subsequent to application of water-immiscible liquids to both the reservoirs and the culture chamber. For example, a reservoir may initially contain the water-immiscible liquid, and an appropriate medium may then be injected into the reservoir below the water-immiscible liquid using e.g. a syringe fitted with a hypodermic needle so that the port of the reservoir chamber is covered with the aqueous medium. A flow of the medium into the culture chamber may then be provided by applying pressure to the upper surface of the water-immiscible liquid in the reservoir chamber. This will force the aqueous medium via the effluent channel into the culture chamber where it will replace a water-immiscible liquid, if present, and cover the port to the effluent channel of the culture chamber. If a water-immiscible liquid is present the desired two-phase system will form. Otherwise a water-immiscible liquid may be applied subsequently, e.g. by overlaying with a pipette.

Once the culture chamber is filled with medium and fitted with the water-immiscible liquid forming the closure, an appropriate cell is placed in the culture chamber. For example, a fertilised oocyte is positioned in the chamber with the aid of a tool, such as a pipette or hypodermic needle. The mesoscale bioreactor platform may also be used for fertilising an unfertilised oocyte. For this purpose the reservoirs may comprise purified or unpurified spermatozoa, or hyaluronidase for cumulus removal from the oocyte. The culture chamber may be fitted with one or more depressions in the bottom surface of the chamber, where each depression is intended for retaining a single fertilised oocyte so that multiple embryos may be cultured simultaneously in the chamber. This allows that multiple cells of the same origin are cultured separately in the same culture chamber. For example, each depression may contain an embryo from the same patient. The bottom of the culture chamber may also be of a conical shape, so that a biological cell or an embryo will be located in the lowest part of the conus. Once the fertilised oocyte is in place in the culture chamber it will then be perfused with appropriate growth media from one or more of the reservoirs. For a fertilised oocyte the culturing period will last for several days, e.g. three days or more, typically up to five days, although longer culturing periods may also be used. The reservoirs should be sufficiently large to supply the culture chamber with the appropriate medium at the relevant flow-rate through-out the culturing period.

Cells contained in the culture chamber will typically be present in the lower layer with the aqueous liquid. An overlay layer of a water-immiscible liquid may also serve to minimise the perfused volume of aqueous liquid in a culture chamber, in addition to preventing evaporation from a growth medium in the aqueous phase and minimising biological contamination of the aqueous liquid. Specifically for IVF-procedures, the application of a layer of a water-immiscible liquid may help maintain pH by preventing evaporation of CO₂.

The chambers of the mesoscale bioreactor platform of the present invention are not limited to a particular shape. However, in a preferred embodiment the shape of the chambers may be generally described as cylindrical with an essentially round circumference. The diameter of this circumference may be larger or smaller than the height of the cylinder. The height of the cylinder will normally follow the vertical axis. In one embodiment the diameter of the cylindrical culture chamber may be from around 2 to around 6 mm, for example around 2.5 mm or 4 mm, and in another embodiment it may be from around 20 to around 30 mm, for example around 25 mm. The depth of these cylindrical culture chambers may be from around 0.5 to around 2 mm, for example around 1.5 mm. Reservoir chambers and waste containers will generally have a greater depth than the culture chambers, typically around 6 mm.

In other embodiments the culture chamber may be generally box-shaped. This box-shape may take the form of a generally flat box with rectangular sides, or the box may be closer to a cube in shape. In one embodiment the culture chamber may be of a width of around 5 to around 10 mm with a length of up to around 50 mm. The depth of such box-shaped culture chambers may be from around 0.5 to around 2 mm.

In one embodiment the mesoscale bioreactor platform of the present invention contains up to 12 cylindrical culture chambers of around 2.5 mm diameter and around 1.5 mm depth each with an optional single depression located in the bottom surface, and the volumes of each of the culture chambers is around 10 μL or less. In this embodiment the culture chambers are connected serially with the reservoirs.

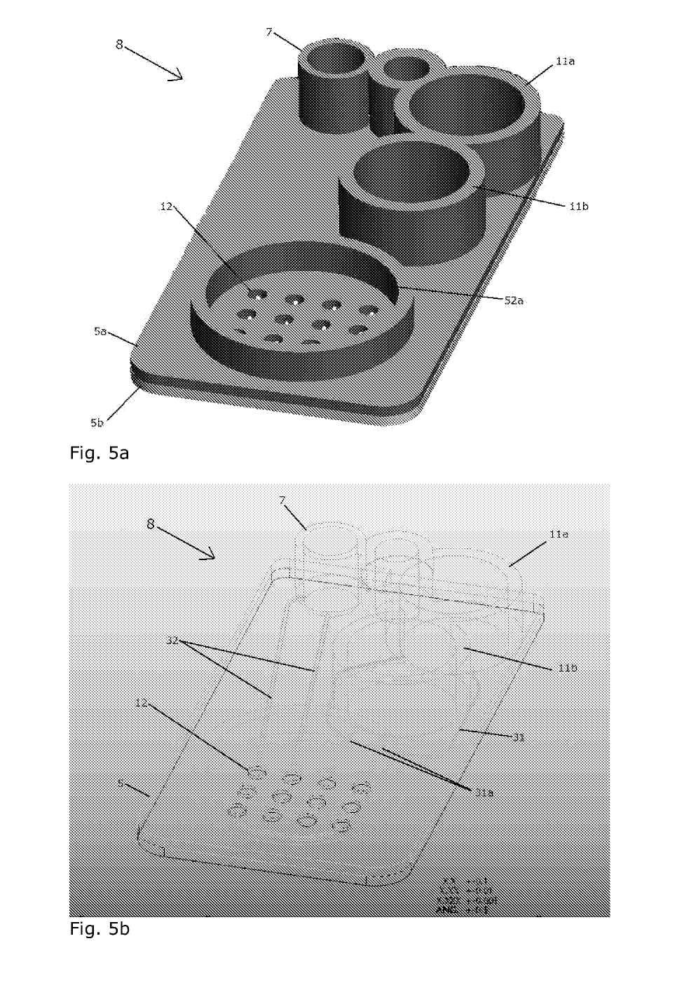

A preferred embodiment of the mesoscale bioreactor platform is illustrated in FIG. 5. This embodiment comprises two reservoir chambers 11a,b of 6 mm depth with respective diameters of 14 mm and 12 mm. Two effluent channels 31 lead from each of the reservoir chambers 11a,b, respectively, to two separate manifolds 31a. From each manifold a channel leads to the first of a series of six culture chambers 12, so that the two groups each of six culture chambers 12 are connected in parallel with the reservoir chambers 11a,b. Each culture chamber 12 has a diameter of 2.5 mm and a depth of 1.5 mm. From each of the last culture chambers 12 in the two series a channel 32 leads to a waste container 7 (of 7.9 mm diameter and 6 mm depth). The culture chambers 12 are arranged in a 3×4 pattern confined within a 25 mm diameter circle. The substrate 5 defines a wall of 4.5 mm height with an inner surface 52a corresponding to the 25 mm circle. This inner surface 52a further defines a well for a water-immiscible liquid, so that the culture chambers 12 will share a single closure formed by the water-immiscible liquid.

In use the two reservoir chambers 11a,b will be filled with appropriate growth media for fertilised oocytes, and the culture chambers 12 will be filled with growth medium from the first of the reservoir 11a or 11b as appropriate. A layer of water-immiscible liquid, such as paraffin oil, will then be provided to each of the reservoir chambers 11a,b, and to the culture chambers as confined by the inner surface 52a, and optionally also to the waste container 7. The thickness of the paraffin oil layer will typically be around 1 mm to around 2 mm. A fertilised oocyte will be placed in each of the optional depressions of each of the culture chambers 12, before starting the culturing by perfusing the culture chambers 12 with growth media from the reservoir chambers 11a,b. This perfusion will be from one reservoir chamber or media from the two reservoir chambers 11a,b may be combined in an appropriate ratio. Throughout the culturing the liquid level in the reservoirs 11a,b may be higher than upper surface of the water-immiscible liquid in the culture chambers 12, which in turn is higher than the liquid level in the waste container 7. However, late in a culturing phase when most or all of the medium has been spent the liquid level in the waste container 7 may approach or even surpass that of the reservoir chambers 11a,b; in this case liquid may be removed from the waste container by aspiration using an externally located pump. It is also possible to increase the pressure above the reservoir chambers 11a,b in order to prevent the liquid flow direction to reverse.

In another embodiment the mesoscale bioreactor platform of the present invention contains one cylindrical culture chamber of approximately 20-40 mm diameter. In this embodiment the culture chamber has 8-20 depressions.

The inner surface of the culture chamber(s) may be smooth or rough, although for certain applications the culture chamber(s) may be fitted with a scaffold supporting cellular growth. Such a scaffold may be part of the material making up the culture chamber(s), or it may be provided in the form of an insertable imprint. The scaffold may be shaped so as to resemble a biologically occurring interface, and it may involve a physically imprinted pattern, or a pattern with a varied pattern of hydrophilic and hydrophobic sites, or a combination of the two. In yet another embodiment the scaffold may be functionalised chemically with species appropriate to cell binding, such as proteins, charged groups, cells, cells debris or the like.

When depressions are present in the culture chamber(s), these may be generally cylindrical with horizontal and vertical dimensions of similar sizes. The dimensions are typically around 500 μm. The depression is suited for retaining the one or more mammalian cells. It is particularly suited for retaining a fertilised oocyte. Thus, prior to the culture of an embryo a fertilised or unfertilised oocyte may be placed, with e.g. a pipette or a hypodermic needle, in the depression in the culture chamber. If only one depression is present it may be generally centrally located in the bottom surface of the culture chamber. If more than one depression are present these may be located along a line in the bottom surface, or they may be laid out in a suitable pattern, such e.g. as that determined by the intersections in a mesh of rectangular, triangular or hexagonal cells, or a mesh similar to a spider's web, or along the perimeters of concentric circles.

The fluidic structures, i.e. channels and the optional manifold, of the mesoscale bioreactor platform may also further comprise a mixing section, which will generally be located between the reservoir chambers and the culture chamber. Thus, the fluid streams from the two or more reservoir chambers may therefore be mixed before the fluid reaches the culture chamber. Such a mixing section may comprise internal structures on an inner surface of a channel section, such as a herring-bone structure or a chaotic mixer, or it may comprise a length contributing element, such as a meander channel or a spiralling channel, allowing the liquids to be mixed by diffusion. The platform may also comprise a manifold or similar structure dividing the flow from the reservoir chambers into a number of channels. The lengths of channels in the bioreactor platform may also be selected in order to control the flow-resistance of the channels, as defined above.

The chambers functioning as reservoirs of the mesoscale bioreactor platform of the present invention are generally of larger volumes than the culture chamber. In a preferred embodiment the volumes of the reservoir chambers are at least 10 times larger than the volume of the culture chamber. In another preferred embodiment the volumes of the reservoir chambers are at least 20 times larger than the volume of the culture chamber.

The reservoir chambers of the mesoscale bioreactor platform may take the form of cylinders, which in one embodiment will be of a sufficient height, such as 6 mm, relative to that of the culture chamber(s) so that the upper surface of the aqueous medium liquid will be at a higher level than the upper surface of the aqueous liquid in the culture chamber(s). The bottom of the cylinder may have a flat surface, or the surface may be conical or funnel-shaped, it may be sloped or of a more complex shape combining the above characteristics.

In one embodiment the mesoscale bioreactor platform is further fitted with a radio frequency identification (RFID)-tag. This RFID-tag may allow a quick and convenient identification of the bioreactor platform. Identification of the bioreactor platform is advantageous when the information contained in the RFID-tag is linked to the identity of a person providing the cells being cultured in a given bioreactor platform.

The chamber of the present invention is preferably formed in a substrate, which in turn may constitute a bioreactor platform. The substrate is preferably one or more thermoplastic polymers, such as poly(methyl methacrylate) (PMMA), cyclic olefin copolymer, or polystyrene (PS), although other materials, such as glass, silicon, metal, elastomeric polymers, or a combination of these may also be used. The materials may be transparent or opaque. Preferably, at least the material constituting the bottom of a culture chamber is transparent. It is further preferred that the substrate materials have a generally hydrophilic surface, and the surface of a naturally hydrophobic material may be modified to make it hydrophilic. Such modification may comprise treating the surface with an oxygen plasma, derivatisation of the surface with charged or hydrophilic moieties, covalent attachment of appropriate silane molecules carrying, e.g. positive or negative charges, or hydrophilic groups. These methods may also be used in combination, and it may also be necessary to provide relevant moieties in a protected form so that the desired functionalities may be obtained after attachment by removing the protective groups. Chemical modification of the surface of a substrate may take place using wet chemical methods or using vapour phase deposition methods; both principles are well-known to those of skill in the art.

In one embodiment, the upper surface of the substrate surrounding the perimeter of the chamber of the invention is oleophobic or superoleophobic. The oleophobicity of the substrate may be characterised by the contact angle between an oil droplet and the surface. The contact angle is defined geometrically as the internal angle formed by a liquid at the three-phase boundary where the liquid, gas and solid intersect. Contact angle values below 90° indicate that the liquid spreads out over the solid surface in which case the liquid is said to wet the solid (this may be termed “oleophilic”). If the contact angle is greater than 90° the liquid instead tends to form droplets on the solid surface and is said to exhibit a non-wetting (or “oleophobic”) behaviour. When the contact angle exceeds 145°, this characteristic may be termed superoleophobic in the context of this invention. Contact angles in excess of 120° will typically require that the surface be provided with a microscale structure prior to being coated with appropriate moieties of desired physico-chemical functionality. A microstructured surface may comprise a random or periodic pattern of structures not exceeding 100 μm in size when seen from above; such a pattern may be provided using e.g. laser ablation, hot embossing, chemical etching or other methods. Depending on the physico-chemical properties of the microstructured surface it may further be necessary to chemically derivatise the surface. Functionalisation of the surface with perfluoro moieties may provide superoleophobic characteristics. Such groups may for example be provided in the form perfluoro silanes applied to the surface in a vapour phase deposition procedure, as is well known in the art.

Channels and chambers of the mesoscale bioreactor platform of the present invention may be formed by joining a first substrate layer comprising structures corresponding to the channels and chambers with a second substrate layer. Thus, the channels are formed between the two substrates upon joining the substrates in layers, and chambers may correspond to the thickness of the layer. The mesoscale bioreactor platform is not limited to two substrate layers. In certain embodiments multiple substrates may be used where each of the substrates may comprise structures for channels and chambers as appropriate. These multiple substrates are then joined in layers so as to be assembled as a mesoscale bioreactor platform.

The structures corresponding to the channels and chambers in the substrates may be created using any appropriate method. In a preferred embodiment the substrate materials are thermoplastic polymers, and the appropriate methods comprise milling, micromilling, drilling, cutting, laser ablation, hot embossing, injection moulding and microinjection moulding. Injection moulding and microinjection moulding are preferred techniques. These and other techniques are well known within the art. The channels may also be created in other substrate materials using appropriate methods, such as casting, moulding, soft lithography etc.

The substrate materials may be joined using any appropriate method. In a preferred embodiment the substrate materials are thermoplastic polymers, and appropriate joining methods comprise gluing, solvent bonding, clamping, ultrasonic welding, and laser welding.

### EXAMPLES

### Example 1

### Construction of a Mesoscale Bioreactor Platform

A prototype mesoscale bioreactor platform consisting of four layers of substrate materials was designed using the 2D drawing software AutoCAD LT (Autodesk, San Rafael, Calif., USA). The bioreactor platform design contained two cylindrical reservoirs of 16 mm diameter and 5 mm depth (1 mL volume), which were connected to a junction by two channels. Each reservoir had a channel allowing to connect the reservoir to the ambient surroundings. A channel from the junction led to three serially connected culture chambers of 4 mm diameter and 1.5 mm depth (similar to a volume of 20 μL). Each culture chamber had a depression of approximately 500 μm diameter and 200 μm in depth in the bottom surface. A waste channel led from the third culture chamber to the ambient surroundings.

The bottom layer of the design of the bioreactor platform was a rectangular plate (5 cm×8 cm size) with a through-hole of 500 μm diameter and the three depressions of the culture chambers. This plate was designed to be joined with a second substrate plate (of 4.5 cm×7.5 cm size) containing three through-holes corresponding to the culture chambers (4 mm diameter) and two additional through-holes (of 500 μm diameter) the positions of which matching the positions of the reservoirs in the above layer. The two 500 μm diameter holes were each connected with a channel (500 μm width) meeting in the junction to form a channel (500 μm width) leading from the junction to the first hole corresponding to a culture chamber; further channels connected the other culture chamber holes, and a final channel was designed to match the through-hole in the bottom layer (thus constituting a waste channel). All channels were designed to be created in the bottom surface of the second layer. A third layer (of 4.5 cm×2.5 cm size) merely contained two 16 mm diameter through-holes corresponding to the reservoirs. The fourth layer (of 4.5 cm×2.5 cm size) contained to channels (500 μm width) leading from positions corresponding to the centres of the two reservoirs to one edge of the plate. The channels of the fourth layer were designed to be created in the bottom surface of this plate.

The AutoCAD LT-designs were used to ablate the structures into substrates of poly(methyl methacrylate) (PMMA) using a Synrad Fenix Marker CO₂-laser (Synrad Inc., Mukilteo, Wash., USA). The transparent PMMA substrates were supplied by Röhm GmbH & Co. (Plexiglas XT20070, Röhm GmbH & Co., Darmstadt, Del.); the layer containing the reservoirs was of 5 mm thickness, all others were of 1.5 mm thickness. Prior to the ablation the AutoCAD LT-designs were converted to encapsulated post-script files and imported into the WinMark Pro software controlling the Synrad Fenix Marker CO₂-laser. Ablation was performed using laser settings which will be well-known to those skilled in the art following an appropriate annealing procedure at 80° C. to prevent stress cracking of the PMMA substrates, the bottom surfaces of the three uppermost substrates were dyed with an IR-absorber dye (ClearWeld LD130, Gentex Corp., Carbondale, Pa., USA). The different layers were then welded together using a Fisba FLS Iron laser scanner (Fisba Optik AG, St. Gallen, CH) capable of yielding a powerful ˜800 nm laser light. Initially the second substrate layer was welded to the bottom substrate layer, and subsequently the third and fourth layers were welded to the growing stack of layers. During the welding the substrates were pressurised appropriately using a vice created with glass that is transparent to the laser light. Optimal laser settings for efficient welding are well known within the art.

The three culture chambers of this prototype mesoscale bioreactor platform are open and accessible, so that a layer of a water-immiscible liquid may be applied to aqueous medium compositions in the chambers.

### Example 2

### Construction of a Mesoscale Bioreactor Platform

A mesoscale bioreactor platform was designed and constructed as described in Example 1 except the three serially connected culture chambers (in the second substrate layer) were replaced with a single culture chamber of 20 mm diameter (volume 0.5 mL). The bottom of this single culture chamber contained six depression (approximately 500 μm diameter and 200 μm depth) placed on the perimeter of a circle of 10 mm diameter located in the centre of the chamber.

### Example 3

### Construction of a Control Unit

A suitable box of a polymeric material was selected to construct a proto-type control unit for housing a mesoscale bioreactor platform. The size of the box was approximately 16×24×12 cm³. The box was fitted with a compartment consisting of a smaller box for containing an aluminium block (approximately 10×7×2 cm³), to function as a heat regulating element, and either of the mesoscale bioreactor platforms described in Example 1 or Example 2. The aluminium block was machined to exactly house the bioreactor platform, and a hole (1 mm diameter) was drilled in it in a location corresponding to the location of the exit of the mesoscale bioreactor platform. The opening of the hole was expanded to house a rubber O-ring (1 mm ID), and the exit hole fitted with a piece of 0.5 mm ID Teflon tube which was connected to micro-scale pH-electrode further being connected to a 2 mL syringe pump. In an alternative design the exit hole was connected to a 360 μm diameter glass capillary. The pH-electrode was connected to a sensor board which was further connected to a PC running LabView (ver. 8, National Instruments, Austin, Tex., USA).

The aluminium block was further machined to house a peltier element which was connected to a DC power supply. An electronic temperature sensor was integrated into the aluminium block. The electronic control for the heating element and the temperature sensor were both connected to the sensor board. A custom made LabView application was created to implement a model predictive control (MPC) algorithm for controlling the temperature on the basis of input from the temperature sensor.

The compartment containing the aluminium block consisted of a transparent plastic box with a closable lid. The bottom side of this box had an approximately 1 cm diameter round hole which was connected to an air supply system capable of supplying the compartment with a laminar air flow surrounding the aluminium block.

The prototype control unit box was further equipped with two syringe pumps each fitted with a piece of tube (0.5 mm ID teflon or 360 μm diameter glass capillary) allowing connection to air inlets of the bioreactor platform, so that a liquid driving force can be provided to the reservoirs to disperse aqueous liquid from the reservoir chambers into a culture chamber.

Control of all pumps was performed from the LabView application via the sensorboard.

### Example 4

### Construction of an IVF-Bioreactor Platform

A bioreactor platform 8 for IVF-procedures containing two reservoir chambers 11a,b and twelve culture chambers 12 was designed and constructed. Perspective views of the bioreactor platform are depicted in FIGS. 5a and 5b showing how each reservoir chamber 11a,b (of 12 and 14 mm diameters, respectively, and 6 mm height) has two effluent channels 31 leading to two separate manifolds 31a. From each manifold a channel leads to the first of a series of six culture chambers 12 (each of 2.5 mm diameter and 1.5 mm depth), so that the two groups each of six culture chambers 12 are connected in parallel with the reservoir chambers 11a,b. From each of the last culture chambers 12 in the two series a channel 32 leads to a waste container 7 (of 7.9 mm diameter and 6 mm depth). The substrate 5 of bioreactor platform 8 defines a wall with an inside surface 52a for confining a layer of water-immiscible liquid (not shown) on the culture chambers 12, so that the culture chambers share a single closure provided with the layer of water-immiscible liquid. The confinement defined by 52a had a diameter of 25 mm and a depth of 4.5 mm.

The bioreactor platform 8 was constructed from two ‘slides’ 5a,b of PMMA, which were joined together in a laser welding procedure. The upper substrate slide 5a was injected moulded from black PMMA and contained structures defining the chambers 11a,b, 7, 12 and channels. The channels were defined in the lower surface of the black PMMA substrate, and the chambers 11a,b, 7, 12 were defined essentially as through-going ‘holes’ in the substrate with ‘walls’ on the upper surface. The lower substrate slide 5b was a transparent PMMA slide of the same size as the upper substrate slide 5a. In locations corresponding to each of the culture chambers 12, a depression was made by laser ablation in the lower substrate slide 5b using the CO₂-laser. The two substrate slides 5a,b were then laser welded together using the laser scanner, so that the channels were formed between the two slides, and so that the lower substrate slide 5b provided a transparent bottom for each of the chambers 11a,b, 7, 12.

### Example 5

### Control of pH by Controlling CO₂-Pressure

An experiment was set up to test control of pH by controlling the CO₂-pressure above a culture chamber with a layer of a water-immiscible layer. Briefly, a system was designed and constructed as explained in the above Examples. The system comprised a single reservoir chamber, a culture chamber and a waste container; a single channel connected the reservoir to the culture chamber, and another channel connected the culture chamber with the waste container. The channel leading from the culture chamber to the waste container comprised a widened section providing room for a pH-electrode. Initially, the reservoir and the culture chambers were filled with X-Vivo medium (Bio Whittaker, Walkersville, Md., USA), and a layer of a water-immiscible layer (a paraffin oil from Sigma-Aldrich, Inc.,) was then applied to each of the chambers. A pH-electrode was placed in the sample port, and an experiment was conducted over a 15 days period. During this period a flow of 1 μL/h was applied from the reservoir chamber via the culture chamber and to the waste.

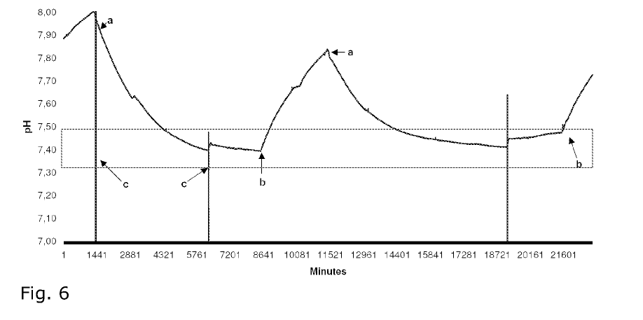

The result of the experiment is presented in FIG. 6 as a curve of pH vs. time. At points labelled “a” a supply of 5% CO₂ in air was applied to the system above the bioreactor, and at points labelled “b” the gas supply was switched back to air without extra CO₂. At the time points labelled “c”, the pH probe was returned to a buffer for recalibration. The box overlaid the plot indicates the normal physiological pH-range of 7.36-7.48.

As can be seen from this experiment it is possible to lower the pH of an aqueous liquid with an overlay layer of a paraffin oil by applying 5% CO₂ in air above the chambers. Upon application of CO₂-free air the pH rose quickly, and thus the combined used of a closure comprising a layer of the paraffin oil with control of the CO₂ pressure allows for a method to retain the pH within the physiological range.

It was further observed in the experiment that the levels of aqueous liquids in the chambers only changed due to transfer of liquids between the chambers via the channel. In other words, there was substantially no evaporation of solvent from any of the chambers.

### Example 6

### Cell Culturing in the System

Two blocks of aluminium were machined to hold the mesoscale bioreactor platform of Example 4 between the two blocks in an appropriately sized enclosure.

The upper aluminium block was machined to house the bioreactor platform, and a hole (1 mm diameter) was drilled in the upper layer block in a location corresponding to the location of the waste container of the mesoscale bioreactor platform. The opening of the hole was expanded to house a rubber O-ring (1 mm ID), and the exit hole fitted with a piece of 360 μm diameter glass capillary tube which was connected to a pH-electrode in a compartment in the lower surface of the upper aluminium block so as to define a sample port. The sample port was further connected to a 2 mL syringe pump. The pH-electrode was connected to a sensor board which was further connected to a PC running LabView (ver. 8, National Instruments, Austin, Tex., USA).

The lower aluminium block was machined to house a heating coil which was connected to a DC power supply. An electronic temperature sensor was integrated into this aluminium block. The electronic control for the heating element and the temperature sensor were both connected to the sensor board. The temperature of the block was controlled using the MPC. The lower aluminium block further comprised a laminar air-flow supply. This consisted of a tube with a horizontal slit (of 1 mm height and 30 mm width) located in a position corresponding to the end of the bioreactor platform with the culturing chambers so as to introduce a horizontal laminar air flow above the culturing chamber with a width similar in size to the width of the chamber. The tube had an entry point (located on the outer surface of the upper aluminium block) for connecting to an air supply, e.g. 5% CO₂ in air. In an alternative design the system employed an integrated gas mixer.

The two aluminium blocks were attached two each other via a hinge mechanism, so that the mesoscale bioreactor platform could be placed on the temperature regulating element in the lower block. By closing the hinge mechanism the Teflon tube in the upper aluminium block would be inserted into the connection chamber on the mesoscale bioreactor platform so as to create a connection for liquid from the culturing chambers to be led to the sample port.

Control of all pumps was performed from a LabView application via the sensorboard.

In use the media reservoirs of the bioreactor platform were filled with growth media for blastocyst culture (medium ‘A’ and ‘B’, respectively), and the cell culturing chamber was primed with medium A. A layer of paraffin oil was then applied to each of the upwards open chambers, and the platform was placed in the aluminium housing. The temperature of the system was set to 37° C., and a flow of 5% CO₂ in air was applied at approximately 1 L/h to the air supply system to equilibrate the growth media in the bioreactor platform and provide a laminar air flow above the open surfaces of the chambers.

On the following day one fertilised oocyte were placed in each of the culturing chambers, and the cell culturing system was closed. A flow of 7.5 μL/h was provided to the culturing chambers in order to apply fresh medium and remove metabolic waste products. Every 1.5 hours 15 μL of waste stream was led to the sample port and the pH was measured. The data processing unit, as represented with LabView applications, monitored the progress of the culturing procedure and recorded the pH and temperature. The MPC algorithm was used to control the temperature to 37° C., and a similar algorithm ensured that the pH was kept within 7.25 to 7.45 by adjusting the flow of the CO₂ in air via the air supply.

The cell culturing procedure lasted 3 days, and the composition of medium, i.e. proportion of A and B medium, was controlled according to a predetermined program. A cell culturing procedure of 5 days duration was also employed with the system.

What is claimed is:

1. A mesoscale bioreactor platform comprising an upwards open chamber for a biological cell, which chamber via a first port is in communication with a first channel for conducting an influent stream of a liquid into the chamber and via a second port is in communication with a second channel for conducting an effluent stream of a liquid away from the chamber, which chamber is provided with a closure comprising a water-immiscible liquid, and wherein said first channel is in fluid communication with a reservoir for a liquid and said second channel is in fluid communication with a waste container.

2. A mesoscale bioreactor platform according to claim 1, wherein the chamber for a biological cell further comprises an aqueous liquid forming a lower phase, wherein the water-immiscible liquid forms an upper phase, and wherein the lower aqueous phase covers the first port and the second port.

3. A mesoscale bioreactor platform according to claim 2, wherein the chamber for a biological cell is perfused with the aqueous liquid.

4. A mesoscale bioreactor platform according to claim 3, wherein the level of the aqueous liquid in the chamber for a biological cell is at a steady state.

5. A mesoscale bioreactor platform according to claim 1, wherein the chamber is formed in a substrate defining a bottom surface and a sidewall of the chamber, which substrate has an upper surface, wherein the upper surface surrounding the perimeter of the chamber is oleophobic or superoleophobic to prevent the water-immiscible liquid from spreading on the upper surface of the substrate.

6. A mesoscale bioreactor platform according to claim 1, wherein the reservoir for one or more of a liquid or the waste container are upwards open chambers.

7. A mesoscale bioreactor platform according to claim 1, wherein the chamber for a biological cell in the bottom surface comprises a depression for retaining the biological cell.

8. A mesoscale bioreactor platform according to claim 1, comprising two or more chambers for a biological cell.

9. A mesoscale bioreactor platform according to claim 1, comprising two or more reservoirs, wherein each reservoir via a channel is in fluid communication with the chamber for a biological cell.

10. A mesoscale bioreactor platform according to claim 1, further comprising a means to provide a liquid driving force to move a liquid via said first channel into the chamber for a biological cell and/or via said second channel away from the chamber for a biological cell.

11. A mesoscale bioreactor platform according to claim 10, wherein the means to provide a liquid driving force is selected from i) dispersing liquid into or aspirating liquid out of the reservoir, the chamber or the waste container using an integrated or external pump, ii) applying a positive relative pressure to the upper surface of a liquid in the reservoir or the chamber, iii) applying a negative relative pressure to the upper surface of a liquid in the chamber or the waste container, iv) adjusting the level of the upper surface of the liquid in the reservoir or the chamber to a higher level than the level of the upper surface of the liquid in remaining ones of the chamber or the waste container relative to a horizontal plane, or any combination of two or more of i), ii), iii), and iv).

12. A mesoscale bioreactor platform according to claim 10, wherein the reservoir, the chamber and the waste container contain an aqueous liquid, and wherein the upper surface of the aqueous liquid in one of the reservoir and the chamber is at a higher level relative to a horizontal plane than the upper surface of the aqueous liquid in one or more of remaining ones of the chamber and the waste container relative to the horizontal plane.

13. A mesoscale bioreactor platform according to claim 1, wherein the volume of the reservoir is at least 10 times larger than the volume of the chamber for a biological cell.

14. A method for modifying the interaction of a content of a chamber with the surroundings comprising the steps of:
providing a mesoscale bioreactor platform according to claim 1;
applying an aqueous liquid to the chamber of the mesoscale bioreactor platform so that the aqueous liquid covers the first and the second ports;
applying a water-immiscible liquid of a density lower than that of water on the aqueous liquid in the chamber to form a lower aqueous phase and an upper phase comprising the water-immiscible liquid;
inducing a flow of an aqueous liquid into said chamber via said first channel and inducing a flow of the aqueous liquid away from said chamber via said second channel.

15. A method according to claim 14, wherein the level of the aqueous liquid in the chamber is maintained at a steady state.

16. A method according to claim 14 further comprising the step of:
controlling the pressure of a gas above the chamber relative to the pressure of the gas originating from the aqueous liquid in the chamber in order to control diffusion of the gas into or out of the aqueous liquid.

17. A method according to claim 16, wherein the gas is CO₂ or O₂.

18. A method of culturing a biological cell comprising the steps of:
providing a mesoscale bioreactor platform according to claim 1;
placing the biological cell in the chamber for a biological cell; and
perfusing the cell with an aqueous liquid.

19. A method according to claim 18, wherein the biological cell is a mammalian, bacterial, yeast, fungal, plant, or insect cell.

20. A method according to claim 19, wherein the mammalian cell is a spermatozoon, oocyte, embryo, stem cell, monocyte, dendritic cell, or a T-cell.

21. A method according to claim 18, wherein the cell is cultured for three days or more.
